(19) United States
(12) Patent Application Publication
(10) Pub. No.: US 2025/0152082 A1
(43) Pub. Date: May 15, 2025

(54) MEASURING A PATIENT'S VISUAL MOTION DETECTION THRESHOLD

(71) Applicant: NeuroAEye, LLC, Louisville, KY (US)

(72) Inventors: Craig E. Andrews, Loudon, TN (US); William V. Padula, Guilford, CT (US); Chris Andrews, Granger, IN (US)

(21) Appl. No.: 18/941,742

(22) Filed: Nov. 8, 2024

Related U.S. Application Data
(60) Provisional application No. 63/597,973, filed on Nov. 10, 2023.

(51) Int. Cl. A61B 5/00

(52) U.S. Cl. CPC A61B 5/4064; A61B 5/7264; A61B 5/7445

(57) ABSTRACT

A patient's visual motion detection threshold is measured by displaying on a display screen a fixation target on a background image. The characteristics of a threshold wave pattern displayed against the background image is adjusted while displaying the fixation target. An indication is received when the threshold wave pattern becomes visible to the patient while adjusting the characteristics of the threshold wave pattern. The patient's measured visual motion detection threshold is established based on receiving the indication.

### CROSS-REFERENCE TO RELATED APPLICATIONS

This claims the benefit of priority to Application No. 63/597,973, filed Nov. 10, 2023, which is incorporated by reference in its entirety.

### FIELD

This relates to the field of vision and, more particularly to visual processing.

### BACKGROUND

Concussion, which is a significant health problem throughout the world, is under-diagnosed. Strokes and neurological disorders display many of the same characteristics as a concussion. Because of that, it is possible to detect many of these conditions in the same manner.

Traditional forms of concussion and neurological testing are not portable and require a significant amount of time to evaluate. For example, the standard instrument for concussion analysis, called the Impact Test, takes about 30 minutes to conduct by a qualified professional in an office setting without any background noise.

### BRIEF SUMMARY

The measurement system, apparatus, and method described here are designed to provide a fast and portable test for detecting physiological conditions that affect a person's visual processing. The measurement is not required to be performed in a medical office. Certain examples of the measurement may even be performed in the field at athletic events or military activities.

An example of the measurement method comprises executing computer program instructions to measure a patient's visual motion detection threshold by displaying on a display screen a fixation target on a background image. The characteristics of a threshold wave pattern displayed against the background image are adjusted while displaying the fixation target. An indication is received when the threshold wave pattern becomes visible to the patient while adjusting the characteristics of the threshold wave pattern. The patient's measured visual motion detection threshold is established based on receiving the indication.

The method may also include one or more of the following features.

The method may further comprise comparing the patient's measured visual motion detection threshold to a pre-defined visual motion detection threshold of the patient.

The patient may have a physiological condition that affects the visual motion detection threshold.

Adjusting the characteristics of the threshold wave pattern displayed against the background image may include varying a contrast of the threshold wave pattern compared to the background image.

Adjusting the characteristics of the threshold wave pattern displayed against the background image may include varying a frequency, amplitude, brightness, type of threshold movement, and/or location of the threshold wave pattern.

The threshold wave pattern may include a sine wave and/or a square wave.

The indication may identify a location on the background image where the threshold wave pattern became visible to the patient.

The indication may identify a time when the threshold wave pattern became visible to the patient.

The patient may be suspected of having a concussion and the method may further comprise comparing the patient's measured visual motion detection threshold to a pre-defined visual motion detection threshold of the patient, thereby confirming whether the patient has a concussion.

The display screen may be on a wearable headset.

An example of a system for measuring a patient's visual motion detection threshold comprises non-transitory memory storing computer program instructions and at least one processor that executes the computer program instructions to display on a display screen a fixation target on a background image. The characteristics of a threshold wave pattern displayed against the background image are adjusted while displaying the fixation target. An indication is received when the threshold wave pattern becomes visible to the patient while adjusting the characteristics of the threshold wave pattern. The patient's measured visual motion detection threshold is established based on receiving the indication.

The system may also include one or more of the following features.

The at least one processor may also execute the computer program instructions to compare the patient's measured visual motion detection threshold to a pre-defined visual motion detection threshold of the patient.

The characteristics of the threshold wave pattern displayed against the background image may be adjusted by varying a contrast of the threshold wave pattern compared to the background image.

The characteristics of the threshold wave pattern displayed against the background image may be adjusted by varying a frequency, amplitude, brightness, type of threshold movement, and/or location of the threshold wave pattern.

The threshold wave pattern may include a sine wave and/or a square wave.

The display screen may be on a wearable headset.

The indication may identify a location on the background image where the threshold wave pattern became visible to the patient.

The indication may identify a time when the threshold wave pattern became visible to the patient.

The at least one processor may also execute computer program instructions to compare the patient's measured visual motion detection threshold to a pre-defined visual motion detection threshold of the patient and confirm whether the patient has a concussion.

An example of an apparatus for measuring a human patient's visual motion detection threshold comprises a display screen and a patient input device in communication with non-transitory memory storing computer program instructions. At least one processor executes the computer program instructions to display on the display screen a fixation target on a background image. The characteristics of a threshold wave pattern displayed against the background image are adjusted while displaying the fixation target. An indication is received when the threshold wave pattern becomes visible to the patient while adjusting the characteristics of the threshold wave pattern. The patient's measured visual motion detection threshold is established based on receiving the indication.

The apparatus may include one or more of the following features.

The at least one processor may also execute the computer program instructions to compare the patient's measured visual motion detection threshold to a pre-defined visual motion detection threshold of the patient.

The characteristics of the threshold wave pattern displayed against the background image may be adjusted by varying a contrast of the threshold wave pattern compared to the background image.

The characteristics of the threshold wave pattern displayed against the background image may be adjusted by varying a frequency, amplitude, brightness, type of threshold movement, and/or location of the threshold wave pattern.

The threshold wave pattern may include a sine wave and/or a square wave.

The apparatus may further comprise a wearable headset carrying the display screen.

The indication may identify a location on the background image where the threshold wave pattern became visible to the patient.

The indication may identify a time when the threshold wave pattern became visible to the patient.

The at least one processor may also execute the computer program instructions to compare the patient's measured visual motion detection threshold to a pre-defined visual motion detection threshold of the patient and confirm whether the patient has a concussion.

The measurement method, system, and apparatus may also include any combination of these features.

### BRIEF DESCRIPTION OF THE DRAWINGS

FIG. 1 is s block diagram of an example of the measurement system.

FIG. 2 is a block diagram of an example of the measurement apparatus.

FIG. 3 is a flow diagram of an example of method of measuring a patient's visual motion detection threshold.

FIG. 4 is a block diagram of how the measurement system measures a patient's visual motion detection threshold.

FIG. 5 is a block diagram of a function of the comparison module.

### DETAILED DESCRIPTION

This disclosure describes certain examples and features, but not all possible examples and features, of the measurement system, apparatus, and method. Where a particular feature is disclosed in the context of a particular example, that feature can also be used, to the extent possible, in combination with and/or in the context of other examples. The measurement, system, apparatus, and method may be embodied in many different forms and should not be construed as limited to only the examples and features described here.

The measurement system, apparatus, and method described herein are used to measure a human patient's visual motion detection threshold while central fixation is maintained on a fixation target for evaluating the just noticeable difference (JND) in visual processing between two visual processes.

The first visual process is the focal process, which identifies detail information and is consciously related to higher perception and cognition. The focal process is handled by the occipital cortex.

The second visual process is called the spatial or ambient process. The spatial process is spatial in function and is a movement detection process. It is used for organization of spatial orientation, posture movement, and balance. The spatial process more globally located in the cortex, midbrain, and brain stem.

The focal process is a slower process and yields to the spatial process so that spatial context can be established before detail information. The spatial process sends information from the peripheral retina of both eyes to the thalamus and midbrain (superior colliculus) where information is matched with the sensorimotor system. This spatial information is then forwarded to 99% percent of the cortex including the occipital cortex before the image of the object reaches this portion of the brain. The purpose is to establish spatial context first and then the focal context applies detail information that will have a spatial base by which to organize relationships. Information from the cortex (occipital, parietal, temporal and frontal lobes) then provides feedback to the spatial process for refinement.

The spatial process is used to sustain preconscious anticipatory matching so that the focal process can attend, fixate, and concentrate. Without the spatial visual process, the focal process would have difficulty releasing from the fixation to shift to a new spatial reference. The system would become stuck and require almost full conscious attention to moving the eyes to another point and even thinking about where to place each foot for sequential steps while attempting to walk.

The human brain processes vision through three pathways:
parvocellular—central “focal” vision, or visual acuity and is consciously controlled;
magnocellular—ambient or motion vision, that is compared by the brain to the proprioceptive system and is subconsciously controlled; and
koniocellular—K cells contribute to brightness contrast information and color contrast.

The magnocellular system is sensitive to motion. When it sends different input than the proprioceptive system, a person may get nauseated or suffer motion sickness.

Usually, following a brain injury or a concussion, the magnocellular pathway is often affected and causes focal binding. The balance between the systems-especially the first two systems fail. The parvocellular system, which is consciously controlled, needs the magnocellular system to calibrate where we are in space and with regard to gravity. This mismatch can cause difficulty with walking, driving, and even moving the head. The parvocellular system takes over since it is conscious and the person becomes disoriented especially in places with sensory overload such as an aisle in a store. The koniocellular system may impede the magnocellular system from working with the parvocellular.

Following a neurological event such as a concussion, a stroke or a beginning neurological disease, the relationship between the bi-modal visual processes can become compromised. The ambient process becomes dysfunctional causing the focal process to become primary without appropriate and accurate spatial context. The focal process isolates on detail and is normally kept from over-emphasis on detail by the spatial process, which is expansive in nature as compared to the focal process, which is isolational.

Without the balance of the spatial process, the focal process becomes overly sensitive to detail and this occurs because the JND of threshold for detail information becomes lowered. Conversely, this produces a condition of higher threshold or JND for the ambient process. This condition has been called Post Trauma Vision Syndrome (PTVS).

Certain examples of the measurement, system, apparatus, and methods evaluate the JND for the two visual processes simultaneously. With a concussion potentially causing PTVS, there will be an increased sensitivity (lowered threshold) for focal detail stimulation and a lessening of sensitivity (higher threshold) for spatial change in the spatial process.

The measurement system, apparatus, and method have several uses. One is to measure the amount of degradation of the neurological system compared to normal. A second use is to allow therapy for those patients to retrain their neurological system (balance their parvo-magno processes) back toward normal.

A third use is detecting glaucoma. Current glaucoma tests involve either static or dynamic testing in many locations. Static testing is performed by a computer. Dynamic testing is typically performed by a technician manually and, therefore, movement speeds are not well-controlled. The measurement system, apparatus, and method provide a dynamic test in many locations to detect changes in contrast, frequency and/or magnitude to test for nerve loss more accurately.

Another use is to train those who wish to become better athletes to better control the balance of their parvo-magno system so as to increase their peripheral awareness for their particular sport. Just as all athletes are not the same in their abilities, much of what makes an elite athlete is their ability to use their senses better than average. Vision, especially peripheral detection of motion, as well as the awareness of others, is something that can be trained. The system, apparatus, and method may include a database of athletes in different sports and positions of those sports. The database may show younger athletes how well they compare to elite athletes using their parvo-magno balance.

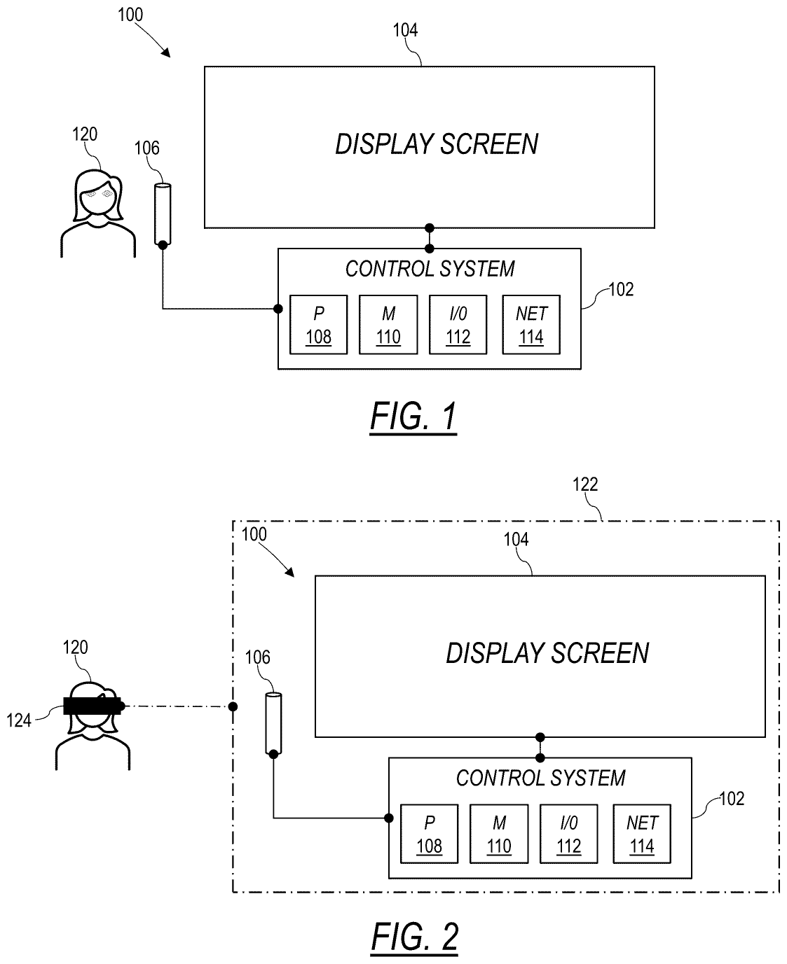

Referring to FIG. 1, an example of the measurement system 100 includes a control system 102, a display screen 104, and a patient input device 106.

The control system 102 is a computing device that includes a processor 108, memory 110, an I/O interface 112, and a network adapter 114. These features may communicate with each other through a bus or wirelessly and may be located within a single device or be divided across multiple devices.

An example of the processor 108 is a computer microprocessor such as one that includes one or more processing units such as a central processing unit (CPU) and a graphical processing unit (GPU). The control system 102 may include one or more of the processors 108. In some cases, one or more of the processors 108 may be accessed remotely relative to one or more of the other processor(s) 108.

An example of the memory 110 includes non-transitory memory containing non-transitory computer executable program instructions. Examples of such memory 110 include a random access memory (RAM), a hard disk, a removable storage device, or remote memory such as cloud storage.

The memory 110 stores data and executable program instructions, such as software programs, for performing various computing functions. The processor 108 is capable of executing the program instructions stored on memory 110 to cause the control system 102 to perform computing operations consistent with the apparatus, system, and method disclosed herein.

An example of the I/O interface 112 includes hardware and software for communication with the control system 102 by a user. The I/O interface 112 may include, for example, a keyboard, mouse, touch screen, camera, microphone, speaker, and the like.

An example of the network adapter 114 includes hardware and software for allowing the control system 102 to communicate information over a network. Examples of the network adapter 114 may include, for example, a local area network (LAN) adapter, a wireless wide area network (WWAN) adapter, a Bluetooth® module, a near field communication adapter, or the like.

The control system 102 is in wired and/or wireless communication with the display screen 104, which is a device that provides a visible output to a user such as, for example, a television screen, a computer screen, an LCD screen, a headset screen, or the like.

The measurement system 100 may be a plurality of independent components in communication or may be combined into an apparatus.

Referring to FIG. 2, the measurement system 100 may be integrated into a measurement apparatus 122 into which the display screen 104, control system 102, and patient input device 106 are integrated.

In certain examples of the measurement system 100 and apparatus 122, the patient 120 wears a headset 124 carrying the display screen 104 and holds or otherwise operates the patient input device 106 while undergoing a visual motion detection threshold measurement. The headset 124 may be configured with a conventional screen, as an extended reality headset, virtual reality headset, and/or an augmented reality headset, for example.

The control system 102 is in wired and/or wireless communication with the patient input device 106 operated by the patient 120 undergoing a visual motion detection threshold measurement.

The patient input device 106 is configured to be operated by the patient 120 to provide electronic input to the control system 102 when the patient is undergoing a visual motion detection threshold measurement. In some examples, the patient input device 106 includes one or more of a joystick, button, microphone, motion detector, camera, or the like that patient 120 operates. The patient input device 106 communicates with the I/O interface 112 and/or the network adapter 114. In other examples, the display screen 104 is a touch screen and the patient input device 106 is the touch screen feature of the display screen 104.

The control system 102 executes program instructions to measure the patient's visual motion detection threshold. The memory 110 stores computer program instructions for performing the measurement. The processor 108 executes the computer program instructions.

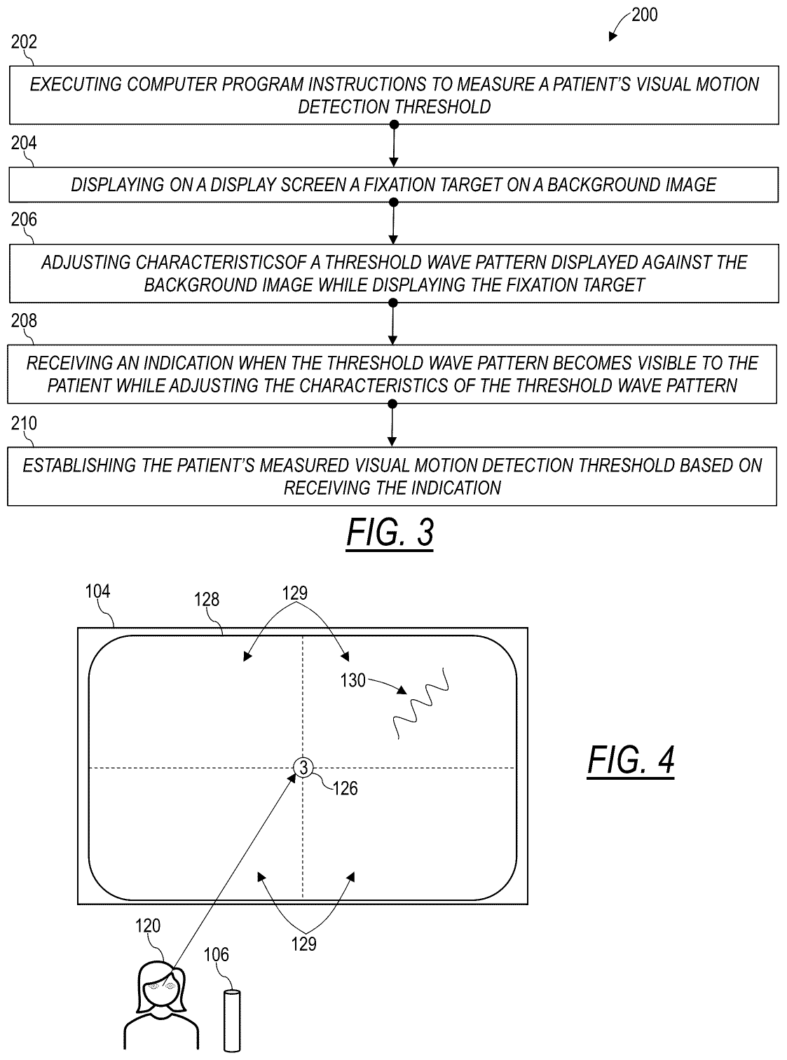

Referring to FIGS. 3 and 4, an example of how the measurement system 100, apparatus 122, and method 200 are used to measure the patient's visual motion detection threshold is now explained.

At block 202, the control system 102 executes program instructions to display on the display screen 104 a fixation target 126 on a background image 128 (block 204). The fixation target 126 is a fixation stimulus presented on the background image 128 in order to create an environment to stimulate focalization.

The fixation target 126 may have many different forms. The fixation target 126 may be, for example, numbers, letters, geometric shapes, and other possibilities. The fixation target 126 may randomly change numbers, letters, geometric shapes, and colors during a measurement. The fixation target 126 is typically displayed centrally on the background image 128 sequentially in a limited zone so as to require the patient to make rapid saccadic fixation movement (rapid movements of the eyes).

The background image 128 may have different forms. In a particular example, the background image 128 is a single color. The background image 128 may divided into four quadrants 129 to provide additional functionality if desired.

On a particular example, the background image 128 is a solid gray image such as a ganzfeld.

In another particular example, the background image 128 may use an optokinetic drum-like movement with solid lines or sinusoidal lines of varying brightness to stimulate the magnocellular pathway and/or contrasting lights and colors to stimulate the koniocellular pathway. Such a background image 128 may work well for testing or training of the visual motion detection threshold while balancing out the control of the parvocellular pathway.

Once the fixation target 126 and background image 128 have been displayed, the patient 120 is instructed to maintain central fixation by focusing their view on the fixation target 126 while being aware of entire spatial field of the background image 128.

One technique for maintaining central fixation on the fixation target 126 is to have the patient 120 call out and identify the fixation target 126 as it changes over time. For example, the patient 120 may call out the numbers or letters presented as the fixation target 126. If the fixation target 126 includes random geometric shapes presented in random colors, each time a geometric shape appears the patient 120 may be required to call out the shape (for the first design presented) and the color of the subsequent shape. This will be repeated for each geometric form presented so that the order will be color-shape-color-shape, with fixed interval of presentation (i.e., one geometric shape per second). The patient 120 must shift their eyes producing a series of saccadic fixations or quick eye movements to fixate on the fixation target 126.

In certain examples, the control system 102 displays four circles in a diamond shape placed centrally on the background image 128. When the measurement begins, one of the circles changes colors. The patient 120 has to touch the fixation target 126 when the circle changes color for the next random circle to change color. The saccades and touch point will drive over-focalization in a concussed patient causing the patient's 120 perception of the threshold wave pattern 130 to be delayed compared to normal.

In certain examples, the patient 120 points toward the fixation target 126 using the patient input device 106 as the fixation target 126 moves over time, requiring the subject to shift their weight and move in different directions with their body and arms during the measurement. If the measurement is performed using an extended reality device, the control system 102 can control what the patient 120 sees and can adjust the field to correct for the patient's eye movements during the measurement.

In some examples, an eye position monitoring system may also be used to verify central fixation.

The fixation target 126 is designed to stimulate the focal process. The threshold wave pattern 130 is used to determine the spatial process JND. Following a concussion causing PTVS, for example, the threshold for spatial change in the peripheral vision serving the spatial process will be elevated causing a decreased sensitivity or awareness to change. This will result in a longer reaction time.

At block 206, the control system 102 executes program instructions to adjust characteristics of a threshold wave pattern 130 displayed against the background image 128 while displaying the fixation target 126.

The threshold wave pattern 130 may randomly appear in one or more of the quadrants 129. The threshold wave pattern 130 may be a sine wave or square wave. The threshold wave pattern 130 may move away or towards the fixation target 126 and may appear in different quadrants 129 of the background image 128.

The characteristics of the threshold wave pattern 130 may be adjusted by varying its frequency, amplitude, brightness, and/or contrast relative to the background image 128. For example, the control system 102 may initially display the threshold wave pattern 130 below the patient's visual motion detection threshold and the visibility is increased until the threshold wave pattern 130 becomes visible to the patient 120.

In some examples, one of the quadrants of the background image 128 will randomly begin to present threshold wave patterns 130, such as sine waves, that begin at zero Hertz (Hz) and develop in one of the outer corners directed toward the center with a high amplitude to low amplitude and a low frequency to a high frequency in the center.

In some examples, the arc of the threshold wave pattern 130 will move outward from the center in one of the quadrants 129 at a rate of at least 2 degrees/sec. Slowly the contrast of the threshold wave pattern 130 will increase until the patient 120 can see the threshold wave pattern 130 and identify the quadrant 129 in which it is located using the patient input device 106. The arc may begin at the center of the display screen 104 in one of the four quadrants 129 or may begin peripherally.

During the measurement, the quadrant 129 in which the threshold wave pattern 130 is displayed may be chosen randomly as well as the start time at which the threshold wave pattern 130 is displayed to reduce anticipation by the patient 120.

The objective of the visual motion detection threshold measurement is to determine when the threshold wave pattern 130 becomes visible to the patient 120 while maintaining central fixation on the threshold wave pattern 130 as the control system 102 adjusts the characteristics of the threshold wave pattern 130. The visual motion detection threshold is the minimum level of visibility of the threshold wave pattern 130 that the patient can visually perceive. The visual motion detection threshold may also be called the just noticeable difference (JND). One particular example of the visual motion detection threshold is a spatial visual process threshold.

To achieve this objective, at block 208, the control system 102 executes program instructions to receive an indication when the threshold wave pattern 130 becomes visible to the patient 120 while adjusting the characteristics of the threshold wave pattern 130. This allows the control system 102 to know when, and under what conditions, the patient first visually perceived the threshold wave pattern 130.

Contemporaneously with the patient 120 first visually perceives the threshold wave pattern 130 in the periphery, the patient 120 immediately provides an indication the patient 120 detected the threshold wave pattern 130. The patient 120 provides the indication on the patient input device 106 and the control system 102 electronically receives the indication.

For example, the patient 120 may press a button, give an audible signal, move a joystick, move a mouse, or tap a touchscreen to provide the indication on the patient input device 106. In certain examples, where the patient input device 106 is a touchscreen or a mouse, the patient 120 may touch the quadrant 129 of the background image 128 where patient 120 visually perceived the threshold wave pattern 130.

At block 210, the control system 102 executes program instructions to establish the patient's 120 measured visual motion detection threshold based on receiving the indication to produce metrics which are stored in memory 110. These metrics include one or more of: the properties of the threshold wave pattern 130 rendered on the display screen 104 prior to the received indication, the timing for the threshold wave pattern 130 rendered on the display screen 104 prior to the received indication, the timing of the received indication, and the properties of the threshold wave pattern 130 at the time of receiving the indication.

These timing metrics represent the patient's JND for being aware of the peripherally presented threshold wave pattern 130 while maintaining central fixation on the fixation target 126. The control system 102 records the time from the start of the measurement to when the patient 120 correctly indicates the quadrant 129 in which the threshold wave pattern 130 becomes visually perceptible to the patient 120.

The metrics may also include information about the threshold wave pattern 130 that was displayed immediately prior to the received indication including one or more of: the type of threshold wave pattern 130, the frequency, amplitude, contrast, brightness, and location of the threshold wave pattern 130 on the background image 128.

The measurement can be performed with both eyes open or with one eye closed or covered.

Different tests may be conducted to determine how wide the subject's magnocellular system is compromised.

The measurement of the patient's visual motion detection threshold may be used to detect physiological conditions that affect the visual motion detection threshold, such as a spatial visual process threshold, for example. Such physiological conditions include, for example, certain neurological conditions such as stroke, head trauma such as concussion, certain infections, drug use, alcohol use, certain environmental conditions, and glaucoma.

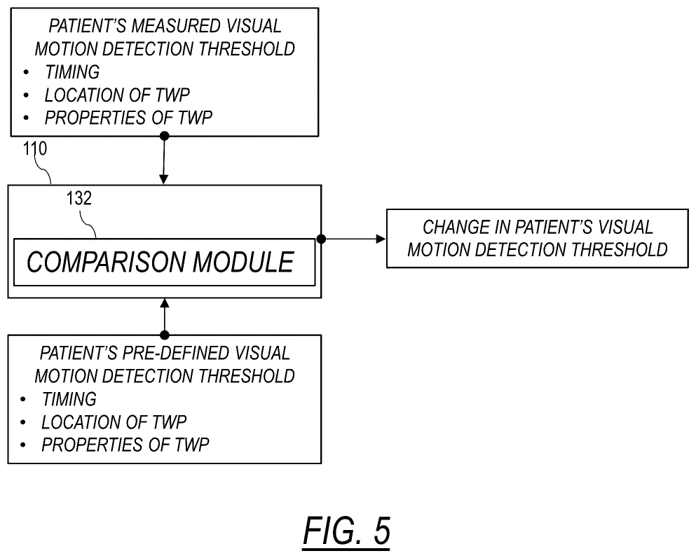

Referring to FIG. 5, to detect physiological conditions that affect the visual motion detection threshold, the control system 102 also executes computer program instructions to compare the patient's measured visual motion detection threshold to a pre-defined visual motion detection threshold of the patient 120 using a comparison module 132 stored on the memory 110. In this context, the patient's pre-defined visual motion detection threshold is measured before the onset of the physiological condition to provide the patient's 120 normal, baseline visual motion detection threshold. The control system 102 stores the patient's predefined visual motion detection threshold metrics in the memory 110. Such metrics may include, for example, the timing of receiving the indication, the location of the threshold wave pattern 130 when the indication was received, and the properties of the threshold wave pattern when the indication was received.

The comparison module 132 is a computer program module that compares the patient's pre-defined visual motion detection threshold to the patient's measured visual motion detection threshold and determines whether the patient's visual motion detection threshold changed. A decrease in the patient's visual motion detection threshold may indicate the patient has a physiological condition that affects the visual motion detection threshold.

If the patient's 120 normal, baseline visual motion detection threshold has not been measured, the control system 102 may use an average normal, baseline visual motion detection threshold for the relevant human population as the patient's pre-defined visual motion detection threshold.

In a particular example, the visual motion detection threshold metrics identify the location of the threshold wave pattern 130 on the background image 128 where the indication was received. The memory 110 stores this information to allow the comparison module 132 to detect changes either over time or compared to a database of physiological conditions.

The memory 110 may include a patient database storing patient identifying information along with each patient's medical and vision records including physiological conditions, acuities, and visual motion detection threshold measurement metrics. As the database grows, the values such as contrast, speed, or thickness of the threshold wave pattern 130 used in the measurements and training may be altered using artificial intelligence software which might determine thresholds which will speed the testing time or enhance the training results. The amounts used may change based on the patient's age, gender, neurological condition, sport, and/or sport position played.

In a particular example, the measurement system 100, measurement apparatus 122, and measurement method 200 are used to determine whether the patient has as a concussion. This can be performed quickly in the field by medical staff on the scene of the event that caused the concussion. For example, the headset 124 may be worn by the patient 120 and the visual motion detection threshold measurement performed. This advantageously provides a much faster, yet reliable, concussion evaluation than the conventional Impact Test.

The measurement system 100, measurement apparatus 122, and measurement method 200 may be used for further testing and/or training of the patient 120. In such examples, the metrics from the indication received from the patient input device 106 during the visual motion detection threshold measurement are recorded in the memory 110. The measurement is repeated and the patient's 120 metrics are compared with the patient's 120 historical metrics using the comparison module 132. This can be performed multiple times to verify results or train the patient 120 to improve the visual motion detection threshold.

In a second measurement, the control system 102 displays a threshold wave pattern 130 on the display screen 104. The threshold wave pattern 130 is above the average visual motion detection threshold of the contrast necessary to be visually perceived by the patient 120 to measure the speed of motion. An arc is displayed moving at about one half degree per second outward from the center (or from a peripheral spot outward). If the patient 120 does not detect the threshold wave pattern 130 or correctly determine which quadrant 129 it is in, the arc begins again one half degree/sec faster. This continues until the patient 120 identifies and indicates two correct quadrant 129 movements in a row.

In a third measurement, the control system 102 displays a threshold wave pattern 130 on the display screen 104. The threshold wave pattern 130 is above the average visual motion detection threshold of the contrast necessary to be visually perceived by the patient 120. An arc that is about one-half the thickness of the arc being used in the second measurement and the visual motion detection threshold measurement is displayed. This arc moves at the same speed as the visual motion detection threshold measurement. The patient 120 will again indicate which quadrant 129 the arc is in. The thickness of the arc will continue to halve until the patient 120 cannot detect the movement.

The measurement system 100 and measurement apparatus 122 can be utilized on the field of an athletic event so that the athlete can be tested for a head injury within minutes of the injury.

The measurement system 100, measurement apparatus 122, and measurement method 200 can also be utilized by medics to test a soldier for a head injury in the field away from a medical facility.

For athletic and military uses, it may be desirable to have measured the patient's pre-defined visual motion detection threshold to have a normal baseline measurement for comparison. The measurement time for the patient's pre-defined visual motion detection threshold may be less than two minutes and can be shortened. Visual motion detection threshold measurements in the field may also be of short duration, such as one to two minutes.

Visual motion detection threshold measurements may be taken a physician in a clinical setting to follow neurological changes such as glaucoma or Alzheimer's.

Using the measurement system 100, measurement apparatus 122, and measurement method 200 to measure a patient's 120 visual motion detection threshold in different locations outside a medical office is a new type of testing.

A person having ordinary skill in the art will understand that the measurement system, apparatus, and method may be modified in many different ways without departing from the scope of what is claimed. The scope of the claims is not limited to only the particular features and examples described above.

That which is claimed is:

1. A method comprising executing computer program instructions to measure a patient's visual motion detection threshold by:
displaying on a display screen a fixation target on a background image;
adjusting characteristics of a threshold wave pattern displayed against the background image while displaying the fixation target;
receiving an indication when the threshold wave pattern becomes visible to the patient while adjusting the characteristics of the threshold wave pattern; and
establishing the patient's measured visual motion detection threshold based on receiving the indication.

2. The method of claim 1, further comprising comparing the patient's measured visual motion detection threshold to a pre-defined visual motion detection threshold of the patient.

3. The method of claim 1, wherein the patient has a physiological condition that affects the patient's visual motion detection threshold.

4. The method of claim 1, wherein adjusting the characteristics of the threshold wave pattern displayed against the background image includes varying a contrast of the threshold wave pattern compared to the background image.

5. The method of claim 1, wherein adjusting the characteristics of the threshold wave pattern displayed against the background image includes varying a frequency, amplitude, brightness, type of threshold movement, and/or location of the threshold wave pattern.

6. The method of claim 1, wherein the threshold wave pattern includes a sine wave and/or a square wave.

7. The method of claim 1, wherein the indication identifies a location on the background image where the threshold wave pattern became visible to the patient.

8. The method of claim 1, wherein the indication identifies a time when the threshold wave pattern became visible to the patient.

9. The method of claim 1, wherein the patient is suspected of having a concussion, the method further comprising:
comparing the patient's measured visual motion detection threshold to a pre-defined visual motion detection threshold of the patient, thereby confirming whether the patient has a concussion.

10. The method of claim 1, wherein the display screen is on a wearable headset.

11. The method of claim 1, wherein the visual motion detection threshold is a spatial visual process threshold.

12. A system for measuring a patient's visual motion detection threshold, the system comprising:
non-transitory memory storing computer program instructions;
at least one processor that executes the computer program instructions to:
display on a display screen a fixation target on a background image;
adjust characteristics of a threshold wave pattern displayed against the background image while displaying the fixation target;
receive an indication when the threshold wave pattern becomes visible to the patient while adjusting the characteristics of the threshold wave pattern; and
establish the patient's measured visual motion detection threshold based on receiving the indication.

13. The system of claim 12, wherein the at least one processor also executes the computer program instructions to compare the patient's measured visual motion detection threshold to a pre-defined visual motion detection threshold of the patient.

14. The system of claim 12, wherein the characteristics of the threshold wave pattern displayed against the background image is adjusted by varying a contrast of the threshold wave pattern compared to the background image.

15. The system of claim 12, wherein the characteristics of the threshold wave pattern displayed against the background image is adjusted by varying a frequency, amplitude, brightness, type of threshold movement, and/or location of the threshold wave pattern.

16. The system of claim 12, wherein the threshold wave pattern includes a sine wave and/or a square wave.

17. The system of claim 12, wherein the display screen is on a wearable headset.

18. The system of claim 12, wherein the indication identifies a location on the background image where the threshold wave pattern became visible to the patient.

19. The system of claim 12, wherein the indication identifies a time when the threshold wave pattern became visible to the patient.

20. The system of claim 12, wherein the at least one processor also executes the computer program instructions to compare the patient's measured visual motion detection threshold to a pre-defined visual motion detection threshold of the patient and confirm whether the patient has a concussion.

21. The system of claim 12, wherein the visual motion detection threshold is a spatial visual process threshold.

22. An apparatus for measuring a patient's visual motion detection threshold, the apparatus comprising a display screen and a patient input device in communication with non-transitory memory storing computer program instructions and at least one processor that executes the computer program instructions to:
display on the display screen a fixation target on a background image;
adjust characteristics of a threshold wave pattern displayed against the background image while displaying the fixation target;
receive an indication from the patient input device when the threshold wave pattern becomes visible to the patient while adjusting the characteristics of the threshold wave pattern; and
establish the patient's measured visual motion detection threshold based on receiving the indication.

23. The apparatus of claim 22, wherein the at least one processor also executes the computer program instructions to compare the patient's measured visual motion detection threshold to a pre-defined visual motion detection threshold of the patient.

24. The apparatus of claim 22, wherein the characteristics of the threshold wave pattern displayed against the background image is adjusted by varying a contrast of the threshold wave pattern compared to the background image.

25. The apparatus of claim 22, wherein the characteristics of the threshold wave pattern displayed against the background image is adjusted by varying a frequency, amplitude, brightness, type of threshold movement, and/or location of the threshold wave pattern.

26. The apparatus of claim 22, wherein the threshold wave pattern includes a sine wave and/or a square wave.

27. The apparatus of claim 22, further comprising a wearable headset carrying the display screen.

28. The apparatus of claim 22, wherein the indication identifies a location on the background image where the threshold wave pattern became visible to the patient.

29. The apparatus of claim 22, wherein the indication identifies a time when the threshold wave pattern became visible to the patient.

30. The apparatus of claim 22, wherein the at least one processor also executes the computer program instructions to compare the patient's measured visual motion detection threshold to a pre-defined visual motion detection threshold of the patient and confirm whether the patient has a concussion.

31. The apparatus of claim 22, wherein the visual motion detection threshold is a spatial visual process threshold.
